(19) United States
(12) Patent Application Publication
(10) Pub. No.: US 2006/0037122 A1
(43) Pub. Date: Feb. 23, 2006

(54) Visor hinged lens system

(76) Inventor: Michael J. McEvily, Victoria, VA (US)

Correspondence Address: Michael McEvily;281-611 LCC, P.O. Box Y, Victoria, VA, 23974, US

(21) Appl. No.: 10/320,760

(22) Filed: Nov. 20, 2002

(51) Int. Cl. A61F 9/00

(52) U.S. Cl. 2/15

(57) ABSTRACT

The Visor Hinged Lens System (VHLS) is an optical accessory that is made of conventional materials, such as plastic, metal, wood, glass, and the like, consisting of an apparatus that is affixed to the visor or brim of a hat or some other headgear, that enables the user to shield his or her eyes; and with a simple adjustment, involving a hinged mechanism, the apparatus can be pushed forward, allowing the user to have unobstructed vision. The VHLS is a unique combination of eyewear that is connected to an article of headwear, in a temporary or a permanent fashion, allowing the user to flip the eye-protecting portion of the apparatus out of the field of vision using a hinged axis, while allowing the same apparatus to be returned to its original position with an opposing motion.

### REFERENCE TO PRIOR ART

The concept of the VHLS is reliant upon three already existing technologies:

1) Visors or Hat Brims;

2) Hinges or Flexible Joints; and

3) Lenses or Eye Protection.

The three essential components of this invention are thus combined in a such a fashion that has never been used before, wherein the combination of these elements has created a completely new device.

The improvement of the forementioned device over all other pre-existing devices to shield one's eyes is that the VHLS is adjustable and allows the user to have an optional shading system (or eye-protection/correction) that is not reliant upon wearing conventional eyeglasses.

The VHLS can be made in a variety of shapes, sizes, and colors to encourage individuality in personal fashion.

### BACKGROUND OF INVENTION

The primary field from which the VHLS originates would be that of optics; however , it can similarly be considered a form of headgear, or clothing accessory.

### SUMMARY OF INVENTION

The VHLS is a form of eye-protection that is not like conventional glasses. It is attached to headgear that is normally worn by the public and has the unique quality of being immediately adjustable for the user. The VHLS can be worn over the eyes, or in an instant, can be pushed out of the visual field with a simple motion that utilizes a hinge that is affixed to the user's headgear. The VHLS is new and fashionable.

### DESCRIPTION OF INVENTION

The VHLS is a form of eye-protection or adornment that is hinged at the upper portion of the frame assembly that would rest against a person's face and upon his or her nose. The VHLS can be fitted with correction lenses, shaded lenses, or could simply be clear plastic or glass. The hinged portion of the lens assembly would be attached to the brim or visor portion of a hat, a cap, or any other similar headgear. The VHLS would be adjustable so that a snug fit could be aceived by any potential user of the product.

The VHLS would be positioned directly in front of a person's eyes and at an instant, by pushing the frame portion of the assembly forward, the VHLS would be pressed flush with the visor or brim of the headgear, out of the visual range of the user. The VHLS is out of immediate view at a push and retrievable with the simple ease of pulling the frame assembly back down into its original position. The Hinge allows this to easily accomplished.

The following claims are entered as dependant claims and as such delineate specific aspects of the same particular product conception: the visor hinged lens system.

1) The VHLS is a new method of protecting one's eyes without the need for conventional glasses or devices that customarily use ear supports or encompassing head-bands.

2) The VHLS is adaptable to any form of headgear, such as a hat, a cap, or anything that is worn on the head.

3) The VHLS can be constructed from conventional materials like plastic, metal, wood, and/or glass using conventional methods of manufacture using standard methods of molding, casting, and other related fabrication techniques.

4) The VHLS can be attached to any headgear in a temporary or permanent manner.

5) The VHLS can be attached to a variety of headgear by connecting it to the visor or brim portion of the hat, cap, etcetera.

6) The VHLS can be attached to any form of headgear using any conventional fastening technique.

7) The VHLS allows for optical adjustability in an instant, by swinging the framed lens portion of the assembly into a forward position, and back down when desired.

8) The VHLS is oriented perpendicularly to the front of a person's face on a lateral axis that can be locked into position to determine the distance from the person's face; once the VHLS is properly oriented to fit the persons individual facial characteristics, it will allow the person to push the frames out of view and into view when desired; the hinged portion of the VHLS is adjustable to suit different size faces that may wear the same headgear.

9) The VHLS will protect a person's eyes while it is in the down position and will be juxtaposed to the person's headgear when it is in the up position.
